(19) United States
(12) Patent Application Publication
(10) Pub. No.: US 2013/0158130 A1
(43) Pub. Date: Jun. 20, 2013

(54) POLYMER COMBINATIONS FOR COSMETIC PREPARATIONS

(71) Applicants: Bjoern Heuer, Hamburg (DE); Bente Nissen, Hamburg (DE); Stefanie Von Thaden, Hamburg (DE); Margaret Horten, Eindhoven (NL); Manuela Koehler, Hamburg (DE)

(72) Inventors: Bjoern Heuer, Hamburg (DE); Bente Nissen, Hamburg (DE); Stefanie Von Thaden, Hamburg (DE); Margaret Horten, Eindhoven (NL); Manuela Koehler, Hamburg (DE)

(21) Appl. No.: 13/700,750

(22) PCT Filed: May 30, 2011
(86) PCT No.: PCT/EP11/58810

§ 371 Date: Feb. 25, 2013

(30) Foreign Application Priority Data
May 31, 2010 (DE) 10 2010 022 061.2

(51) Int. Cl. A61K 8/88; A61Q 19/00; A61K 8/73; A61K 8/81; A61K 8/87

(52) U.S. Cl. CPC A61K 8/88; A61K 8/8111; A61K 8/87; A61K 8/731; A61K 8/8176; A61Q 19/00
USPC 514/772.1; 514/772.6

(57) ABSTRACT

The invention comprises dermatological or cosmetic preparations comprising a triple combination of anionic polymer and two rheology modifying polymers which increase the viscosity of the preparation by at least 200% at a high shear rate and do not increase the viscosity at a low shear rate by more than 50%, thus imparting an improved sensory richness during spreading, in comparison to preparations without this combination.

The invention comprises dermatological or cosmetic preparations, in particular based on an oil-in-water emulsion, which are thickened and/or stabilized with at least one anionic polymer, preferably with an acrylic acid-based polymer. Additionally, the preparations comprise at least two further rheology-modifying polymers which increase the viscosity of the preparation at high shear by at least 200% and the viscosity at low shear by not more than 50%.

The combination of anionic thickeners and at least two rheology-modifying polymers leads to the cosmetic or dermatological preparations having a sensory richness upon spreading compared with the preparations without this combination.

Skin care products, specifically in the form of lotions or creams for the entire body, aim to supply the skin with moisture in the long term, strengthen the skin barrier and keep the skin smooth, soft and supple. Dry skin has a tendency toward visible and feelable flakes and is characterized by a low water and lipid content in the stratum corneum. Skin care products therefore firstly comprise water-binding compounds, such as e.g. glycerol (“moisturizer”) and secondly a series of lipids in order to strengthen the natural skin barrier. The higher the fractions of water-binding substances and/or lipids, the more marked the skin care aspect.

Products which are commercially available therefore differ very considerably in their fractions of moisturizers and lipids according to skin type.

Besides the skin care aspect, the lipid fraction in particular influences the sensory properties of the products to a considerable degree. A high fraction of lipids leads to a more marked, greasy/oily residue on the skin, but also provides for particular richness during spreading. The consumer associates richness with the aspect of care and also skin pampering.

Particularly in the field of standard body lotions which are designed for normal to slightly dry skin, a relatively small amount of lipids suffices for caring for the skin. Often, the products are applied daily, e.g. after showering, and the consumer expects the product to absorb rapidly with little greasy/oily residue on the skin. In order to be able to offer this light skin feel, these lotions are often formulated with distinctly less than 10% of light lipids; products for particularly dry skin do not uncommonly contain more than 20% lipids. As a consequence, these products also feel less rich upon spreading, they are more watery.

It is therefore of particular interest to also make the lotions for normal to slightly dry skin more sensorily attractive upon spreading, in particular to increase the richness, without losing the light skin feel after spreading.

Increasing the richness simply by increasing the fraction of lipids, however, leads to the formation of an oily/greasy residue on the skin and is detrimental to the light skin feel.

Polymers are generally only used in skin care preparations, in particular lotions, for stabilizing the emulsion and/or for establishing the viscosity. Polymers based on acrylic acid, which are generally crosslinked and neutralized with a suitable base, are widespread.

This class of rheology modifiers is called “Alkali Swellable Emulsions” (ASE), the INCI name is “Carbomer”. Hydrophobically modified variants, which have a greater interaction with the emulsifiers and lipids, are also used. The term used here is “Hydrophically modified Alkali Swellable Emulsions (HASE), the INCI in most cases being “Acrylates/C10-30 Alkylacrylates Crosspolymer”. Other classes of rheology-modifying polymers are also known, e.g. “Hydrophobically modified Ethoxylated Polyurethanes” (HEUR) or “Associative Cellulose Thickeners” (ACT), but these are less widespread in lotions on account of sensory disadvantages or higher raw material costs.

Polymers of the HEUR class are used particularly when compatibility problems prevent the use of the classic ASE or HASE thickeners, e.g. in cationic systems (incompatibility to the anionic ASE/HASE thickener), or formulations with a low pH. As a result of their associative properties, they interact with hydrophobic formulation constituents such as lipids and surfactants, for which reason the structure buildup in these systems is often favored.

Product data sheets from Rohm & Haas from 1994 concerning the polymer Aculyn 44, which is a typical polymer of the HEUR class, contain emulsion formulations with Aculyn 44, e.g. in combination with UV filters, alpha-hydroxy acids, antiperspirants, peroxides or cationic constituents.

A further class of polymers for thickening cosmetic formulations are crosslinked polyvinylpyrrolidones (PVP). These are water-swellable and nonionic, for which reason they are likewise more likely to be used in systems which are difficult to thicken on account of compatibility problems with ionic formulation constituents. Example formulations with such polymers can be found e.g. in patent specifications of ISP from 1992.

U.S. Pat. No. 5,716,634 describes aqueous gels with crosslinked vinyllactam polymers. WO 199207011 describes formulations with crosslinked PVP, and EP 1105092 discloses formulations which can comprise a crosslinked PVP as thickener. U.S. Pat. No. 6,024,942 discloses photoprotective formulations which can comprise a crosslinked PVP as thickener.

It is therefore desirable to provide cosmetic or dermatological skin care preparations which, on the one hand, convey to the user a sensorily rich feel without altering the fraction of lipids and thus, on the other hand, reducing and/or avoiding the disadvantages of rich preparations.

It has been found that the object is achieved if at least two further rheology-modifying polymers are added to a cosmetic or dermatological preparation, in particular based on an oil-in-water emulsion which is stabilized and/or thickened with at least one polymeric anionic thickener, such that the viscosity of the preparation at low shear (measured using a rotational rheometer with the name Rheomat R 123 from proRheo GmbH with measuring body 1) increases by less than 50%, but at high shear (at 300 Pa, measured using a shear-stress-controlled, air-mounted rheometer SR-2000 from Rheometric Scientific, plate/plate 25 mm, 1 mm gap, shear stress ramp from 0 to 400 Pa at 40 Pa/min and 32° C.) increases by at least 200%.

The cosmetic or dermatological preparation according to the invention comprises at least one polymeric anionic thickener and additionally at least two further rheology-modifying polymers.

The fraction of rheology-modifying polymers is preferably to be selected in the range of up to 5% by weight, in particular up to 2% by weight, preferably up to 1% by weight, based on the total mass of the preparation.

According to the invention, the use of at least two rheology-modifying polymers in cosmetic or dermatological preparations comprising at least one polymeric anionic thickener leads to the viscosity of the preparation being increased at high shear by at least 200% and the viscosity at low shear being increased by not more than 50%.

Simply increasing only the viscosity of a skin care preparation, i.e. not only at high shear, but also at low shear, no longer produces a lotion, but a cream. This cream does then feel richer, but is not as pleasant to apply and spread as a lotion.

Only by means of the special combination of the rheology-modifying polymers, in particular in a fraction below 5% by weight, particularly preferably in a fraction below 2% by weight, with at least one polymeric anionic thickener are the advantageous rheological properties achieved. The preparation obtainable in this way is a lotion from the point of view of texture and can be applied to the skin like one but, upon spreading (at a higher shear), it exhibits an increased viscosity and thus a richer feel.

The rheology-modifying polymers according to the invention are particularly advantageously used in a total fraction of less than 5% by weight, preferably less than 2% by weight, particularly preferably less than 1% by weight (active polymer content), based on the total weight of the formulation. Although the rheology-modified polymers are used in these relatively small quantitative fractions, the viscosity of the preparation at high shear is increased by at least 200% but is increased by not more than at most 50% at low shear. According to the invention, it is particularly advantageous in this connection if these at least two further rheology-modifying polymers are used in total with up to 1% by weight (active polymer content), based on the total weight of the formulation.

Rheology-modifying polymers according to the invention are to be selected from the group of the polymers based on vinylpyrrolidone, the associative polyurethanes (HEUR), the associative cellulose thickeners (ACT), the polyamides of the classes ATPA (tertiary amide terminated polyamides), ETPEA (ester terminated poly(ester-amides), PAOPA (polyalkyleneoxy terminated polyamides) and PEPA (polyester polyamides), of the polyolefins or of the styrene block copolymers (SBC).

Polymers preferred according to the invention here are polymers based on vinylpyrrolidone as monomer, particularly preferably those which consist exclusively of vinylpyrrolidone and very particularly preferably if they are of high molecular weight or additionally crosslinked. Such polymers are available e.g. under the trade names PVP K-90 or PVP K-120 from ISP. Crosslinked variants are known e.g. under the name Kollidon® CL from BASF or ACP-1120 from ISP.

Rheology-modifying polymers likewise preferred according to the invention are associative polyurethanes (HEUR). These are preferably linear reaction products of optionally different diisocyanates with optionally different diols, where preferably polyalkylene glycols which preferably have hydrophobic (associative) end groups can be used as diol components. Aculyn® 44 or 46 from Rohm & Haas or Luvigel® Star from BASF, for example, can be used as associative polyurethanes suitable according to the invention.

Rheology-modifying polymers likewise preferred according to the invention are associative cellulose thickeners (ACT). These are chemically modified celluloses which are modified with ethylene glycol and long-chain alcohols. Representatives of this class are e.g. cetyl hydroxyethylcelluloses (e.g. a number of Natrosol® grades from Hercules), or C12-16 Alkyl Hydroxyethyl Ethylcellulose (e.g. Elfacos® CDHM from Akzo Nobel).

Rheology-modifying polymers likewise preferred according to the invention are polyamides of the classes ATPA (tertiary amide terminated polyamides), ETPEA (ester terminated poly(ester-amides), PAOPA (polyalkyleneoxy terminated polyamides) and PEPA (polyester polyamides), such as e.g. Sylvaclear® A200V, C75V, AF1900V, WF1500V, PA1200V or PE400V from Arizona Chemical.

Rheology-modifying polymers likewise preferred according to the invention are polyolefins or styrene block copolymers (SBC). The polyolefins are e.g. polyisobutene (PIB). The SBC is e.g. AB, ABA, (AB)n or similar block copolymers, where one block is polystyrene and the other block is a polyolefin (co)polymer, such as e.g. polyisoprene, polybutadiene, ethene/propene copolymer, ethene/butene copolymer, etc.

Good results have been achieved as regards the required rheology properties with all of the listed rheology-modifying polymers. Very particularly good results are exhibited by polymers based on vinylpyrrolidone (PVPs), associative polyurethanes (HEURs) and associative cellulose thickeners (ACTs), which are therefore to be used with particular preference.

Preferably, at least two different rheology-modifying polymers are added to the preparations.

Polymeric anionic thickeners according to the invention that are to be used are those substances which comprise a polymer which comprises acrylic acid as one monomer. This is intended to mean polymers which fall under the class of the ASE (alkali swellable emulsion) or HASE (hydrophobically modified alkali swellable emulsion) thickeners. As well as acrylic acid, these thickeners can comprise further, preferably nonionic, monomers.

As stabilizing and thickening polymer, the formulations according to the invention therefore preferably comprise a polymer which comprises acrylic acid or methacrylic acid from the class of the ASE (alkali swellable emulsion) thickeners or HASE (hydrophobically modified alkali swellable emulsion) thickeners.

Of particular suitability here are homopolymers of acrylic acid with the INCI name Carbomer (e.g. Carbopol® grades from Lubrizol), copolymers of acrylic acid with alkyl acrylates with the INCI name Acrylates Copolymer (e.g. Aculyn® 33 from Rohm & Haas), copolymers of acrylic acid with vinylpyrrolidone with the INCI name Acrylic Acid/VP Crosspolymer (Ultrathix® P-100 from ISP), copolymers of acrylic acid with C₁₀-C₃₀ alkyl acrylates with the INCI name Acrylates/C10-30 Alkyl Acrylates Crosspolymer (Carbopol® or Pemulen® grades Lubrizol or Synthalen® grades from 3V Sigma), copolymers of acrylic acid with ethyl acrylate and associative alkyl acrylates with the INCI name Acrylates Copolymer (Carbopol Aqua SF-1® from Lubrizol) or copolymers of (meth)acrylic acid with alkyl acrylates and ethoxylated hydrophobically modified alkyl acrylates with the INCI names Acrylates/Steareth-20 Methacrylate Copolymer, Acrylates/Beheneth-25 Methacrylate Copolymer, Acrylates/Steareth-20 Methacrylate Crosspolymer (Aculyn® 22, 28 or 88 from Rohm & Haas) or the INCI Acrylates/Palmeth-25 Acrylates Copolymer (Synthalen® from 3V Sigma).

Polymeric anionic thickeners according to the invention which can likewise be selected are thickeners based on acrylamidomethylpropanesulfonic acid (AMPS), such as e.g. ammonium acryloyldimethyltaurate/VP copolymer (Aristoflex® AVC from Clariant) or ammonium acryloyldimethyltaurate/beheneth-25 methacrylate copolymer (Aristoflex® HMB from Clariant).

It may be advantageous if mixtures of two or more polymeric anionic thickeners according to the invention are used.

Within the context of the invention, it is useful to use active ingredients for positively influencing aging skin which reduce the formation of wrinkles and also existing wrinkles. Particularly preferred active ingredients are therefore bioquinones, in particular ubiquinone Q10, isoflavone and isoflavonoids, genistein, arctiin, cardiolipin, lipoic acid, antifreezing proteins, hop extracts and hop-malt extracts, and/or substances which promote the restructuring of connective tissue, isoflavonoids and isoflavonoid-containing plant extracts such as e.g. soya and clover extracts, which can also be used very readily in the preparations and skin covering matrices according to the invention.

It has also been found that the combination product according to the invention is particularly suitable to use active ingredients for supporting the skin functions in dry skin, such as, for example, vitamin C, biotin, creatine, creatinine, propionic acid, glycerol, green tea extracts, white tea extracts or solutions.

In a similar way, the incorporation of active ingredients for alleviating and/or positively influencing irritative skin conditions, be it sensitive skin in general or skin irritated by noxae (UV light, chemicals), has proven to be advantageous. Active ingredients to be mentioned here are sericosides, various extracts of licorice, licochalcone A, silymarin or silyphos, flavonoids, dexpanthenol, ethanol, inhibitors of the prostaglandin metabolism, in particular of the cyclooxygenase, and of the leukotriene metabolism, in particular of the 5-lipoxygenase, but also of the 5-lipoxygenase inhibitor protein, FLAP.

The incorporation of modulators of pigmentation has also proven to be advantageous. Mention is to be made here of active ingredients which reduce the pigmentation of the skin and thus lead to a cosmetically desired lightening of the skin and/or reduce the appearance of age spots and/or lighten existing age spots, such as tyrosine sulfate, vitamin C, lipoic acid and liponamide, various extracts of licorice, kojic acid, hydroquinone, arbutin, fruit acids, in particular alpha-hydroxy acids (AHAs), bearberry (Uvae ursi), ursolic acid, ascorbic acid, green tea extracts, aminoguanidine and/or pyridoxamine.

In a similar way, active ingredients which bring about an increased/more rapid tanning of the skin (advanced glycation end products (AGE), lipofuscins, nucleic acid oligonucleotides, purines and pyrimidines, NO-releasing substances, whether with or without the influence of UV light, have proven to be suitable active ingredient additives in the preparations according to the invention.

Active ingredients that are likewise suitable for the use are, for example, folic acid, phytoene, urea, D-biotin, coenzyme Q10, flavone glycosides such as e.g. α-glucosylrutin, carnitine, polydocanol, natural and synthetic isoflavonoids, in particular genistein, flavonoids, carotenoids, creatine, creatinine, taurine, ascorbic acid, and derivatives thereof, oxygen, tocopherol, and esters thereof, dihydroxyacetone, 8-hexadecene-1,16-dicarboxylic acid, long-chain hyaluronic acids (i.e. with an average molecular weight of 1 million to 3 million Daltons) and short-chain hyaluronic acids (i.e. with an average molecular weight of 5000 to 1 million Daltons), licochalcone A and mixtures thereof. Ingredients of this type can each be present in an amount of from 0.01 to 30% by weight, based on the total weight of the preparation.

Formulations according to the invention which comprise one or more antiwrinkle active ingredients, such as flavone glycosides, in particular α-glycosylrutin, coenzyme Q10, retinol and esters thereof, vitamin E and derivatives thereof, and also other antiwrinkle active ingredients known to the person skilled in the art, are suitable in particular for protecting against esthetically unattractive changes in the skin, as arise, for example, in the case of tired skin or in the case of skin aging. These changes include e.g. dryness, roughness and the formation of dryness wrinkles, itching, reduced regreasing especially after washing, visible vascular dilations such as teleangiectases or couperosis, flaccidity and formation of lines and wrinkles, local hyper-, hypo- and malpigmentations such as age spots, increased susceptibility to mechanical stress such as cracking and other changes known to the person skilled in the art. Furthermore, the emulsions according to the invention are advantageously suitable for combating the appearance of dry and/or rough skin.

Furthermore, the use of one or more antioxidants in formulations according to the invention is advantageous. Suitable antioxidants which can be selected are one or more substances from the group comprising amino acids and derivatives thereof such as e.g. glycine, histidine, tyrosine and tryptophan, imidazoles and derivatives thereof, such as e.g. urocanic acid, peptides and derivatives thereof, such as e.g. D,L-carnosine, D-carnosine, L-carnosine and anserine, carotenoids, carotenes and derivatives thereof such as e.g. α-carotene, β-carotene and lycopene, lipoic acid and derivatives thereof such as e.g. dihydrolipoic acid, aurothioglucose, propylthiouracil and other thiols, and salts thereof such as e.g. thioredoxin, glutathione, cysteine, cystine, cystamine, and the glycosyl, N-acetyl, methyl, ethyl, propyl, amyl, butyl, lauryl, palmitoyl, oleyl, γ-linoleyl, cholesteryl and glyceryl esters thereof, dilauryl thiodipropionate, distearyl thiodipropionate, thiodipropionic acid and derivatives thereof such as esters, ethers, peptides, lipids, nucleotides, nucleosides and salts, sulfoximine compounds such as e.g. buthionine sulfoximines, homocysteine sulfoximine, buthionine sulfones, penta-, hexa- and heptathionine sulfoximine in very low tolerated doses (e.g. pmol to μmol/kg), also (metal) chelating agents such as e.g. α-hydroxy fatty acids, palmitic acid, phytic acid and lactoferrin, α-hydroxy acids such as e.g. citric acid, lactic acid and malic acid, humic acid, bile acid, bile extracts, bilirubin, biliverdin, EDTA, EGTA and derivatives thereof, unsaturated fatty acids and derivatives thereof such as e.g. γ-linolenic acid, linoleic acid and oleic acid, folic acid and derivatives thereof, ubiquinone and ubiquinol and derivatives thereof, vitamin C and derivatives thereof, such as e.g. ascorbyl palmitate, Mg ascorbyl phosphate and ascorbyl acetate, tocopherols and derivatives thereof such as e.g. vitamin E acetate, vitamin A and derivatives thereof, such as e.g. vitamin A palmitate, coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof such as e.g. ZnO and ZnSO4, selenium and derivatives thereof such as e.g. selenomethionine, stilbenes and derivatives thereof such as e.g. stilbene oxide and trans-stilbene oxide, and also the derivatives of said active ingredients that are suitable according to the invention such as salts, esters, ethers, sugars, nucleotides, nucleosides, peptides or lipids.

The amount of antioxidants (one or more compounds) in the formulations according to the invention is preferably 0.001 to 30% by weight, particularly preferably 0.05 to 20% by weight, in particular 0.1 to 10% by weight, based on the total weight of the preparation.

If vitamin E or derivatives thereof are the antioxidant or the antioxidants, it is advantageous to select their respective concentrations from the range from 0.001 to 10% by weight, based on the total weight of the preparation. If vitamin A, vitamin A derivatives, carotenes or derivatives thereof are the antioxidant or the antioxidants, it is advantageous to select their respective concentrations from the range from 0.001 to 10% by weight, based on the total weight of the preparation. It is particularly advantageous if the cosmetic emulsions according to the present invention comprise cosmetic active ingredients, preferred active ingredients being antioxidants which are able to protect the skin against oxidative stress.

Within the context of the invention, it is further advantageous if substances for improving the microbiological stability, in particular substances with an antimicrobial effect, in particular against bacteria, yeasts and fungi, are used. In this connection, those to be mentioned with preference are benzoic acid, its esters and salts, propionic acid and its salts, salicylic acid and its salts, 2,4-hexadienoic acid (sorbic acid) and its salts, formaldehyde and paraformaldehyde, 2-hydroxydiphenyl, zinc pyrithione, inorganic sulfites and bisulfites, chlorobutanolum, 4-hydroxybenzoic acid, its salts and esters, 3-acetyl-6-methyl-2,4(3H)-pyrandione (dehydracetic acid) and its salts, formic acid and its sodium salt, 1,6-bis(4-amidino-2-bromophenoxy)-n-hexane (dibromohexamidine) and its salts, ethylmercury(II)-thiosalicylic acid, sodium salt (thiomersal), phenylmercury and its salts, 10-undecylenic acid and its salts, 5-amino-1,3-bis(2-ethylhexyl)-5-methylhexahydropyrimidine (hexetidinum), 5-bromo-5-nitro-1,3-dioxane, 2-bromo-2-nitro-1,3-propanediol (bronopol), 2,4-dichlorobenzyl alcohol, N-(4-chlorophenyl)-N′-(3,4-dichlorophenyl)urea (triclocarban), 4-chloro-m-cresol, 2,4,4′-trichloro-2′-hydroxydiphenyl ether (triclosanum), 4-chloro-3,5-dimethylphenol, 1,1′-methylenebis (3-(1-hydroxymethyl-2,5-dioximidazolidin-4-yl)urea (imidazolidinylurea), poly(hexamethylenediguanide) hydrochloride, 2-phenoxy-ethanol, hexamethylenetetramine, 1-(3-chloroallyl)-3,5,7-triaza-1-azoniaadamantane chloride, 1-(4-chlorophenoxy)-1-(imidazol-1-yl)-3,3-dimethyl-2-butanone, 1,3-bis(hydroxymethyl)-5,5-dimethyl-2,4-imidazolidinedione, benzyl alcohol, 1-hydroxy-4-methyl-6-(2,4,4-trimethylpentyl)-2-pyridone and its monoethanolamine salt, 2,2′-methylenebis(6-bromo-4-chlorophenol) (bromochlorophen), 3-methyl-4-(1-methylethyl)phenol, mixture of 5-chloro-2-methyl-3(2H)-isothiazolone and 2-methyl-3(2H)-isothiazolone with magnesium chloride and magnesium nitrate, 2-benzyl-4-chlorophenol (chlorophenum), 2-chloroacetamide, chlorhexidine, its acetate, gluconate and hydrochloride, 1-phenoxypropan-2-ol, N-alkyl (C12-C22)trimethylammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxymethylurea, 1,6-bis(4-amidinophenoxy)-n-hexane (hexamidinum) and its salts, glutaraldehyde (pentane-1,5-dial), 5-ethyl-1-aza-3,7-dioxabicyclo (3.3.0)octane, 3-(4-chlorophenoxy)-1,2-propanediol (chlorophenesin), sodium hydroxymethylamonoacetate, silver chloride, benzethonium chloride, benzalkonium chloride, bromide and saccharinate, benzyl hemiformal, iodopropynyl butylcarbamate (IPBC), 3-iodo-2-propynyl butylcarbamate, 2-methyl-3-(2H)-isothiazolone (methylisothaizolinones), piroctone olamine and ethyl lauroyl arginate. Methylisothiazolinones, the combinations of ethyl lauryl arginate with phenoxyethanol, benzethonium chloride with phenoxyethanol or piroctone olamine with phenoxyethanol are particularly advantageous.

Moreover, it is advantageous according to the invention if substances are used which protect the skin against UV radiation, this radiation being reflected or absorbed by the substances. Preferably, ethylhexyl triazone, diethylhexyl-butamidotriazone, 2-ethylhexyl methoxycinnamate, octyl methoxycinnamate, ethylhexyl salicylate, octyl salicylate, homosalate, octocrylene, isoamyl p-methoxycinnamate, benzophenone-3, benzophenone-2, benzophenone-4, phenylbenzimidazole sulfonic acid, polysilicone-15, butyl methoxydibenzoylmethane, diethylamino hydroxybenzoyl hexyl benzoate, disodium phenyl dibenzimidazole tetrasulfonate, methylene bis-benzotriazolyl tetramethylbutylphenol, bis-ethylhexyloxyphenol methoxyphenyl triazine, titanium dioxide, zinc oxide, or others can be used here. Combinations of 2 or more representatives of these substances in any desired form are likewise advantageous here within the context of the invention.

Moreover, the formulations according to the invention can comprise further ingredients known in cosmetic formulations, such as e.g. acetyl trifluoromethylphenyl valylglycine, acrylamide/ammonium acrylate copolymer, acrylates/C12-22 alkylmethacrylate copolymer, aluminum/magnesium hydroxide stearate, ammonium lactate, ammonium polyacrylate, ammonium polyacryloyldimethyl taurate, arginine PCA, beerwax, benzethonium chloride, capryloyl salicylic acid, β-carotene, cinnamic acid, coco glucoside, copper gluconate, dehydroxanthan gum, diphenyl dimethicone, disodium adenosine triphosphate, disodium succinate, disteardimonium hectorite, dodecene, Eperua falcate, hydrogenated palm glycerides citrate, hydrogenated palm kernel glycerides, hydrolyzed wheat protein, PG-propyl methylsilanediol, casein, kinetin, hydroxyethyl acrylate/sodium acryloyldimethyltaurate copolymer, isodeceth-6, linseed acid, lutein, lyopene, magnesium aspartate, melibiose, oxo-thiazolidinecarboxylic acid, palmitoyl pentapeptide 4, PEG-8 laurate, PG-10 stearate, phenethyl alcohol, phenylpropanol, polyacrylate-13, polyacrylate-3, quinic acid, sarcosine, saxifrage sarmentosa extract, Scutellaria baicalensis extract, shikimic acid, sodium metabisulfite, soy isoflavone, tocopheryl glucoside, trideceth-6, zeaxanthin, zinc gluconate, triacetrin, 1,2-hexanediol, hydroxyethylpiperazine ethane sulfonic acid, nicotinamide, penethyl alcohol, penthylene glycol, carnauba wax, chlorhexidine digluconate, oleyl erucate, acetyltrifluoromethylphenylvalylglycine, acrylamide ammonium acrylate copolymer, aluminum magnesium hydroxide stearate, ammonium lactate, ammonium polyacrylate, ammonium polyacryloyldimethyltaurate, arginine PCA, capryloylsalicylic acid ester, cinnamic acid, cocoglucoside, copper gluconate, diphenyldimethicone, disodium adenosine triphosphate, disodium succinate, disteardimonium hectorite, dodecene, eperua falcate, hydrogenated palm glyceride, hydrogenated palm glyceride citrate, hydrogenated palm kernel glycerides, hydrolyzed wheat protein PG-propylmethylsilanediol, hydroxyethyl acrylate/sodium acryloyldimethyltaurate copolymer, isodeceth-6, linseed acid, magnesium aspartate, melibiose, oxothiazolidinecarboxylic acid, palmitoyl pentapeptide 4, PEG-8 laurate, phenethyl alcohol, phenylpropanol, polyacrylate-13, polyacrylate-3, sarcosine, saxifrage sarmentosa extract, Scutellaria baicalensis extract, sodium metabisulfite, soya isoflavone, tocopheryl glucoside, trideceth-6, zinc gluconate, triacetin, 1,2-hexanediol, hydroxyethylpiperazine ethane sulfonic acid, nicotinamide, penethyl alcohol, penthylene glycol, carnauba wax, chlorhexidine digluconate, oleyl erucate, provided the required viscosity features are retained.

Moreover, the use of substances for positively influencing the odor of the formulation is useful. These include e.g. dipropylene glycol, methyl dihydrojasmonate, phenethyl alcohol, linalool, linalyl acetate, 2,6-dimethyl-7-octen-2-ol, alpha-hexyl-cinnamaldehyde, 2-acetonaphthone-1,2,3,4,5,6,7,8-octahydro-2,3,8,8-tetramethyl p-t-butyl-alpha-methyldihydrocinnamic aldehyde, benzyl acetate, 1,3,4,6,7,8-hexahydro-4,6,6,7,8,8-hexamethylcyclopenta-gamma-2-benzopyran, methyl cedryl ketone, ethylene brassylate, 4-(4-hydroxy-4-methylpentyl)-3-cyclohexene-1-carboxyaldehyde, benzyl salicylate, hexyl salicylate orange oil, alpha-isomethylionone, diethyl phthalate, 4-t-butylcyclohexyl acetate, patchouli oil 3,7-dimethyl-2,6-octadien-1-ol, tetrahydrolinalool, hydroxycitronellal, isopropyl myristate, 3,7-dimethyl-6-octen-1-ol, orange terpenes, heliotropin, terpinyl acetate, omega-pentadecalactone, methyl-alpha-ionone, lavandin oil, lemon oil, bergamot oil, 7-acetyl-1,1,3,4,4,6-hexamethyltetralin, coumarin, ethyllinalool, amyl salicylate, 2-tert-pentyl-cyclohexyl acetate, 3-methyl-5-phenyl-1-pentanol, cedrol, benzyl benzoate, vanillin, alpha-amylcinnamaldehyde, dimethyl phthalate, d-limonene, 2-isobutyl-4-hydroxy-4-methyltetrahydropyran, triethyl citrate, terpineol, lavender oil, diethylene glycol monoethyl ether, 2-phenoxyethyl isobutyrate, anisyl alcohol, 3-pentyltetrahydro(2H)pyranyl acetate, methyl ester of rosin, partially hydrogenated, isobornyl acetate, rosemary oil, petitgrain oil, 1,4-dioxacyclohexadecane-5,16-dione, isoamyl salicylate, gamma-undecalactone, alpha-ionone, oxacyclohexadecen-2-one, 7-octen-2-ol, 2-methyl-6-methylene, dihydro derive. 1,2-propylene glycol, 3-(5,5,6-trimethylbicyclo(2.2.1)hept-2-yl)cyclohexan-1-ol, geranium oil, musk ketone, cedrenyl acetate, isobornylcyclohexanol, ionone, benzyl alcohol, gamma-nonalactone, i-menthol, cyclohexyl salicylate, dihydromyrcenyl acetate, citral, orange terpenes (natural), cedarwood oil, alpha-pinene, majantol, phenoxyethanol, ethyl acetate, cedrol methyl ether, 1,5,9-trimethyl-13-oxabicyclo(10.1.0)trideca-4,8-diene, peppermint oil, eugenol, ethyl maltol, benzaldehyde, cinnamic alcohol, 3,7-dimethyl-1-octanol, alpha-methyl-3,4-methylene dioxyhydrocinnamic aldehyde, beta-pinene, d-camphor, methyl abietate, cedryl acetate, ylang ylang oil, sandalwood oil, mineral oil, dimethyl benzyl carbinyl butyrate, ethyl butyrate, geranyl acetate, hexylene glycol, myrcene, alpha-methylonantheme, beta-ionone, 3-(4-t-butylphenyl)propanal, 3,7-dimethyloctan-3-yl acetate, acetic acid, (1-oxopropoxy)-1-(3,3-dimethylcyclohexyl), eucalyptol, 4-carvomenthenol, stearic acid, menthanyl acetate, eucalyptus oil, dihydroterpinyl acetate, o-t-butylcyclohexyl acetate, isoeugenol, alpha-terpineol, cyclamen aldehyde, hydroxycitronellol, myrcenyl acetate, nopyl acetate, 3,7-dimethyl-1,3,6-octatriene, rhodinol, dimethyl benzyl carbinyl acetate, tricyclodecenyl propionate, 2-methyl-5-phenylpentan-1-ol, sclareoate, 3-isocamphyl cyclohexanol, trans-anethole, hexahydro-4,7-methanoinden-5(6)-yl acetate, 4-(p-hydroxyphenyl)-2-butanone, nerolidol, alpha-butylcinnamaldehyde, bornyl acetate, ethyl methylphenylglycidate, trans-beta-ionone, camphene, juniper berry oil, mandarin oil, nutmeg oil, spearmint oil, grapefruit oil, labdanum oil, galbanum oil, menthone, trichloromethyl phenyl carbinyl acetate, alpha-methylbenzyl acetate, ethyl-2-methyl-1,3-dioxolane-2-acetate, 2,6-nonadienal, abietyl acetate, anisic acid, diphenyl ether, triacetin, 2-methyl-4-phenyl-2-butanol phenylethyl acetate, 1-phenyl-3-methyl-3-pentanol, anisyl acetate, cinnamic aldehyde, p-methylanisole, 5-phenylpentanol, diethyl malonate, citronellal, nerol, undecanal, 2-methyl-, hexyl alcohol, glyceryl caprylate, methyl 2-nonenoate, octyl acetate, decanal, lauryl alcohol, lauric aldehyde, ethyl vanillin, 3-phenyl-1-propanol, octanal, butylated hydroxytoluene, 4-acetyl-6-t-butyl-1,1-dimethylindane, delta-3-carene, benzyl laurate, neryl acetate, ethyl acetoacetate, hexyl acetate, menthol liquid, citronellyl acetate, tetrahydromyrcenol, diacetin, menthyl acetate, 3(4),8(9)-dihydroxymethyl tricyclo(5.2.1.0 (2,6)decane, 2,4-dimethyl-3-cyclohexen-1-carboxaaldehyde, cedrenol, phenylacetaldehyde glyceryl acetal, sabinene, 3,7,11-trimethyl-1,2,10-dodecatrien-3-ol (cis & trans), octyldodecanol, formaldehyde cyclododecyl ethyl acetal myristicin, 3,7-dimethyl-2(3),6-nonadienenitrile, ethyllinylyl acetate, 2-methylbutyl acetate, cis-3-hexenyl salicylate, 2-methyl-4-(2,6,6-trimethyl-2(1)-cyclohexen-1-yl)butanal, maltol isobutyrate, 2-methyl-3(4-(2-methylpropyl)phenyl)propanal, 12-oxahexa-decanolide, 1,1-dimethoxy-2,25-trimethyl-4-hexene, 1,6,7,8-tetrahydro-1,4,6,6,8,8-hexamethyl-as-indacen-3<2H>-one, bergamot oil, bergaptene free, treemoss abs., citrus oil distilled, lemon terpenes, gamma-decalactone, 2-methyl-4-phenyl-2-pentanone, allyl phenoxyacetate, methyl-delta-ionone, citronella oil, clove bud oil, thyme oil, lime oil, bois de rose oil, cognac oil, neroli bigarade oil, spike lavender oil, vetiver oil, fir needle oil, methylpentenolone, lemon oil terpenes, isobutyl salicylate, beta-caryophyllene, pulegone, thymol, gamma-terpinene.

The preparations according to the invention preferably comprise at least one of the active ingredients described above. Particular preference is given here to selecting skin-moisturizing or skin-barrier-strengthening active ingredients.

According to the invention, skin care preparations is the term used to refer to those preparations which donate moisture to the skin and/or protect its barrier effect. In particular, the preparations according to the invention are therefore skin care preparations.

Preferably, the preparations according to the invention are based on an oil-in-water emulsion.

The difference between a cream and lotion is its particular viscosity; below 10 000 mPas the term used is a lotion; above this the term used is a cream.

To measure the viscosity at low shear, the rotational rheometer with the name Rheomat R 123 from proRheo GmbH with the measuring body 1, often used as standard for cosmetic compositions, is used. It is a routine measurement which is carried out at room temperature.

At high shear, the viscosity is by means of a shear-stress-controlled, air-mounted Rheometer SR-2000 from Rheometric Scientific, plate/plate 25 mm, 1 mm gap, shear stress ramp from 0 to 400 Pa at 40 Pa/min. In order that this measurement reflects as well as possible the real operation during spreading on the skin, the skin temperature is measured.

Within the context of the invention, room temperature is defined as 25° C., skin temperature as 32° C.

The viscosity η is defined as the ratio of shear stress T and shear rate in accordance with DIN 53019. I.e. the stated viscosity increases refer to the dynamic viscosity η [1 Ns/m²=1 Pas].

Two different principles play a part for the viscosity measurements.

The values measured using the rheometer at high shear are varied over the shear stress. The shear rate is defined as shear stress/viscosity, i.e. in turn a shear of for example 10s⁻¹ can arise at different shear stresses. For all of the preparations according to the invention, the value for the shear rate is at least 300 s⁻¹ since the viscosity at 300 Pa was always below 1 Pas.

The Rheomat measures at low shear constantly at about 10 s⁻¹.

According to the invention, therefore, a high shear is preferably referred to as at least 300 s⁻¹ and a low shear rate in the range 10 s⁻¹.

The examples below are to describe the invention in more detail without having a limiting effect. The quantitative data refer to fractions by weight, unless disclosed otherwise.

TABLE 1
Base formulation #1

| Ingredient | % by wt. |
|---|---|
| Cetearyl Alcohol | 0.8 |
| Sorbitan Stearate | 0.8 |
| Glyceryl Stearate Citrate | 2.4 |
| Octyldodecanol | 1 |
| Caprylic/Capric Triglyceride | 4 |
| Propylparaben | eq. |
| Myristyl Myristate | 3 |
| Phenoxyethanol | eq. |
| Dimethicone | 2 |
| Glycerin | 9 |
| Sodium Hydroxide | eq. |
| Carbomer | 0.3 |
| Alcohol | 3 |
| Aqua | ad 100 |
| Methylparaben | eq. |
| Parfum | eq. |

At least two further, rheology-modifying polymers were incorporated into the base formulation #1. Table 2 shows their influence on the viscosity at low and high shear.

TABLE 2
Influence of the rheology-modifying polymers
(polymer 1 and 2) in base formulation #1

| | | Visc. 1 | Visc. 2 |
|---|---|---|---|
| Polymer 1 | Polymer 2 | [%]* | [%]** |
| Aculyn 44¹⁾ | Polyisobutene (0.5%) | 45% | 600% |
| (0.5% active) | | | |
| Aculyn 44¹⁾ | Sylvaclear AF1900V⁴⁾ | 50% | 1000% |
| (0.5% active) | (0.5%) | | |
| Aculyn 44¹⁾ | Sylvaclear WF1500V⁵⁾ | 50% | 1300% |
| (0.5% active) | (0.5%) | | |
| Aculyn 46²⁾ | Sylvaclear WF1500V⁵⁾ | 10% | 250% |
| (0.2% active) | (0.5%) | | |
| Aculyn 44¹⁾ | ACP-1120 (0.35%)³⁾ | 25% | 350% |
| (0.35% active) | | | |
| Aculyn 44¹⁾ | Polyisobutene (0.5%) | 20% | 350% |
| (0.35% active) | | | |
| ACP-1120³⁾ (0.35%) | Elfacos CDHM⁶⁾ (0.25%) | 45% | 300% |
| ACP-1120³⁾ (0.25%) | Elfacos CDHM⁶⁾ (0.35%) | 40% | 350% |
| ACP-1120³⁾ (0.35%) | Polyisobutene (0.5%) | 5% | 250% |
| ACP-1120³⁾ (0.35%) | Sylvaclear PA1200V⁷⁾ | 20% | 400% |
| | (0.35%) | | |

*Increase in the viscosity at low shear: measurement by Rheomat R 123 in % compared with the base formulation.
**Increase in the viscosity at high shear (300 Pa, plate/plate 25 mm, 1 mm gap, 0 to 400 Pa at 40 Pa/min) in % compared with the base formulation.
¹⁾INCI PEG-150/Decyl Alcohol/SMDI Copolymer
²⁾INCI PEG-150/Stearyl Alcohol/SMDI Copolymer
³⁾INCI Vinylpyrrolidone Crosspolymer (prop.)
⁴⁾INCI Polyamide-3
⁵⁾INCI Polyamide-3
⁶⁾INCI C12-16 Alkyl Hydroxyethyl Ethylcellulose (prop.)
⁷⁾INCI Polyamide-3

TABLE 3
Base formulation #2

| Ingredient | % by wt. |
|---|---|
| Cetearyl Alcohol | 1 |
| Glyceryl Stearate | 2.4 |
| Isopropyl Palmitate | 3.4 |
| Sodium Cetearyl Sulfate | 0.1 |
| Propylparaben | eq. |
| Dimethicone | 1 |
| Glycerin | 13 |
| Sodium Hydroxide | eq. |
| Carbomer | 0.3 |
| Alcohol | 3 |
| Aqua | ad 100 |
| Methylparaben | eq. |
| Parfum | eq. |

At least two further, rheology-modifying polymers were incorporated into the base formulation. Table 4 shows their influence on the viscosity at low and high shear.

TABLE 4
Influence of the rheology-modifying
polymers in base formulation #2

| | | Visc. 1 | Visc. 2 |
|---|---|---|---|
| Polymer 1 | Polymer 2 | [%]* | [%]** |
| ACP-1120³⁾ (0.35%) | Elfacos CDHM⁶⁾ (0.25%) | 40% | 350% |
| ACP-1120³⁾ (0.25%) | Elfacos CDHM⁶⁾ (0.35%) | 45% | 350% |
| ACP-1120³⁾ (0.35%) | Polyisobutene (0.5%) | 15% | 250% |
| ACP-1120³⁾ (0.35%) | Aculyn 44¹⁾ (0.35% active) | 40% | 400% |

*Increase in the viscosity at low shear: measurement by Rheomat R 123 in % compared with the base formulation.
**Increase in the viscosity at high shear (300 Pa, plate/plate 25 mm, 1 mm gap, 0 to 400 Pa at 40 Pa/min) in % compared with the base formulation.
¹⁾INCI PEG-150/Decyl Alcohol/SMDI Copolymer
²⁾INCI PEG-150/Stearyl Alcohol/SMDI Copolymer
³⁾INCI Vinylpyrrolidone Crosspolymer (prop.)
⁴⁾INCI Polyamide-3
⁵⁾INCI Polyamide-3
⁶⁾INCI C12-16 Alkyl Hydroxyethyl Ethylcellulose (prop.)
⁷⁾INCI Polyamide-3

TABLE 5
Base formulation #3

| Ingredient | % by wt. |
|---|---|
| Glyceryl Stearate Citrate | 2 |
| Octyldodecanol | 4 |
| Dicaprylyl Ether | 4 |
| Stearyl Alcohol | 2 |
| Octocrylene | 5 |
| Bis-Ethylhexyloxyphenol | 2 |
| Methoxyphenyl Triazine | |
| Butyl Methoxydibenzoylmethane | 5 |
| Hydrogenated Coco-Glycerides | 2 |
| Dicaprylyl Carbonate | 1 |
| Phenoxyethanol | eq. |
| C12-15 Alkyl Benzoate | 7 |
| VP/Hexadecene Copolymer | 1 |
| Tocopheryl Acetate | 0.1 |
| Aqua | ad 100 |
| Glycerin | 8 |
| Sodium Hydroxide | eq. |
| Acrylates C10-30 Alkyl | 0.2 |
| Acrylates Crosspolymer | |
| Xanthan Gum | 0.3 |
| Titanium Dioxide + | 3 |
| Trimethoxycaprylylsilane | |
| Alcohol Denat. | 5 |
| Ethylparaben | eq. |
| Methylparaben | eq. |
| Propylparaben | eq. |
| Ethylhexylglycerin | eq. |
| Parfum | eq. |

At least two further, rheology-modifying polymers were incorporated into the base formulation. Table 4 shows their influence on the viscosity at low and high shear.

TABLE 6
Influence of the rheology-modifying
polymers in base formulation #3

| | | Visc. 1 | Visc. 2 |
|---|---|---|---|
| Polymer 1 | Polymer 2 | [%]* | [%]** |
| ACP-1120³⁾ (0.35%) | Elfacos CDHM⁶⁾ (0.25%) | 30% | 300% |
| ACP-1120³⁾ (0.25%) | Elfacos CDHM⁶⁾ (0.35%) | 40% | 300% |
| ACP-1120³⁾ (0.35%) | Polyisobutene (0.5%) | 10% | 300% |
| ACP-1120³⁾ (0.35%) | Aculyn 44¹⁾ | 30% | 350% |
| | (0.35% active) | | |

*Increase in the viscosity at low shear: measurement by Rheomat R 123 in % compared with the base formulation.
**Increase in the viscosity at high shear (300 Pa, plate/plate 25 mm, 1 mm gap, 0 to 400 Pa at 40 Pa/min) in % compared with the base formulation.
¹⁾INCI PEG-150/Decyl Alcohol/SMDI Copolymer
²⁾INCI PEG-150/Stearyl Alcohol/SMDI Copolymer
³⁾INCI Vinylpyrrolidone Crosspolymer (prop.)
⁴⁾INCI Polyamide-3
⁵⁾INCI Polyamide-3
⁶⁾INCI C12-16 Alkyl Hydroxyethyl Ethylcellulose (prop.)
⁷⁾INCI Polyamide-3

All of the formulations given in tables 2, 4 and 6 were assessed sensorily by a panel of experts (10 trained subjects) as being richer than the respective base formulations from tables 1, 3 and 5.

The preparations according to the invention behave during application like a lotion and during spreading like a cream.

The preparations according to the invention thus exhibit a richer feel for the user merely by adding at least two rheology-modifying polymers without having to increase the lipid content or undertake other changes to the formulation.

The viscosity increase by at least 200% at high shear therefore means an increase in the richness upon spreading.

The viscosity increase by less than 50% at low shear means that texture and/or solidity of the lotion does not change; it thus does not become a cream, but remains a lotion.

The lotion differs compared to the corresponding base formulation without the additional rheology-modifying polymers merely by virtue of richer sensory properties upon spreading. The texture and other characteristic of the lotion is not influenced, and the skin feel after rubbing in remains as light as with the corresponding base formulation, which describes in particular the amount of residue and the feel of the complete absorption of the formula into the skin. The user, who has no requirement for a particularly intense supply of lipids on account of a normal skin state, and ensures during his/her skin care that the product absorbs completely into the skin and does not leave behind any oily or greasy residues, is provided, by virtue of the preparations according to the invention, with a sensorily considerably more attractive product since it does not feel watery, but rich and creamy upon spreading without having the mentioned negative influence on ability to absorb and amount of residue.

1-10. (canceled)

11. A cosmetic or dermatological preparation, wherein the preparation comprises at least one polymeric anionic thickener and at least two further rheology-modifying polymers.

12. The preparation of claim 11, wherein the at least two further rheology-modifying polymers increase the viscosity of the preparation at high shear by at least 200% and the viscosity of the preparation at low shear by not more than 50%.

13. The preparation of claim 11, wherein the preparation comprises not more than 5% by weight of the at least two further rheology-modifying polymers, based on a total weight of the preparation.

14. The preparation of claim 13, wherein the preparation comprises not more than 2% by weight of the at least two further rheology-modifying polymers.

15. The preparation of claim 13, wherein the preparation comprises not more than 1% by weight of the at least two further rheology-modifying polymers.

16. The preparation of claim 11, wherein the at least two further rheology-modifying polymers comprise at least two polymers selected from polymers based on vinylpyrrolidone; associative polyurethanes (HEUR); associative cellulose thickeners (ACT); polyamides of the classes ATPA (tertiary amide terminated polyamides), ETPEA (ester terminated poly(ester-amides), PAOPA (polyalkyleneoxy terminated polyamides), and PEPA (polyester polyamides); polyolefins; and styrene block copolymers (SBC).

17. The preparation of claim 11, wherein the at least two further rheology-modifying polymers comprise at least two polymers selected from polymers based on vinylpyrrolidone; associative polyurethanes (HEUR); and associative cellulose thickeners (ACT).

18. The preparation of claim 11, wherein the at least two further rheology-modifying polymers comprise at least two polymers selected from PEG-150/Decyl Alcohol/SMDI Copolymer, PEG-150/Stearyl Alcohol/SMDI Copolymer, Vinyl-pyrrolidone Crosspolymer, Polyamide-3 and C12-16 Alkyl Hydroxyethyl Ethyl-cellulose.

19. The preparation of claim 11, wherein the at least one polymeric anionic thickener comprises one or more polymers comprising acrylic acid units and/or methacrylic acid units.

20. The preparation of claim 19, wherein the at least one polymeric anionic thickener comprises one or more polymers selected from ASE (“Alkali Swellable Emulsion”) thickeners and HASE (“Hydrophobically modified Alkali Swellable Emulsion”) thickeners.

21. The preparation of claim 11, wherein the preparation further comprises at least one active ingredient selected from skin-moisturizing active ingredients and skin-barrier-strengthening active ingredients.

22. The preparation of claim 11, wherein the preparation is based on an oil-in-water emulsion.

23. The preparation of claim 11, wherein the preparation comprises at least one polymeric anionic thickener selected from polymers comprising acrylic acid units and/or methacrylic acid units, and not more than 2% by weight, based on a total weight of the preparation, of at least two rheology-modifying polymers comprising at least two polymers selected from polymers based on vinylpyrrolidone; associative polyurethanes (HEUR); associative cellulose thickeners (ACT); polyamides of the classes ATPA (tertiary amide terminated polyamides), ETPEA (ester terminated poly(ester-amides), PAOPA (polyalkyleneoxy terminated polyamides), and PEPA (polyester polyamides); polyolefins; and styrene block copolymers (SBC), and wherein the at least two further rheology-modifying polymers increase the viscosity of the preparation at high shear by at least 200% and the viscosity of the preparation at low shear by not more than 50%.

24. The preparation of claim 23, wherein the at least one polymeric anionic thickener comprises one or more polymers selected from ASE (“Alkali Swellable Emulsion”) thickeners and HASE (“Hydrophobically modified Alkali Swellable Emulsion”) thickeners.

25. The preparation of claim 23, wherein the at least two further rheology-modifying polymers comprise at least two polymers selected from polymers based on vinylpyrrolidone; associative polyurethanes (HEUR); and associative cellulose thickeners (ACT).

26. The preparation of claim 24, wherein the at least two further rheology-modifying polymers comprise at least two polymers selected from PEG-150/Decyl Alcohol/SMDI Copolymer, PEG-150/Stearyl Alcohol/SMDI Copolymer, Vinyl-pyrrolidone Crosspolymer, Polyamide-3 and C12-16 Alkyl Hydroxyethyl Ethyl-cellulose.

27. The preparation of claim 23, wherein the preparation is based on an oil-in-water emulsion.

28. A method of increasing the viscosity of a cosmetic or dermatological preparation comprising at least one polymeric anionic thickener, wherein the method comprises incorporating in the preparation at least two further rheology-modifying polymers which increase the viscosity of the preparation at high shear by at least 200% and at low shear by not more than 50%.

29. A method of increasing the richness of a cosmetic or dermatological preparation comprising at least one polymeric anionic thickener upon spreading without increasing the proportion of lipids, wherein the method comprises incorporating in the preparation at least two further rheology-modifying polymers which increase the viscosity of the preparation at high shear by at least 200% and at low shear by not more than 50%.

30. The method of claim 29, wherein the at least one polymeric anionic thickener is selected from polymers which comprise acrylic acid units and/or methacrylic acid units and the at least two rheology-modifying polymers comprise at least two polymers selected from polymers based on vinylpyrrolidone; associative polyurethanes (HEUR); associative cellulose thickeners (ACT); polyamides of the classes ATPA (tertiary amide terminated polyamides), ETPEA (ester terminated poly(ester-amides), PAOPA (polyalkyleneoxy terminated polyamides), and PEPA (polyester polyamides); polyolefins; and styrene block copolymers (SBC).
